(19) United States
(12) Patent Application Publication
(10) Pub. No.: US 2018/0267338 A1
(43) Pub. Date: Sep. 20, 2018

(54) TEMPERATURE-SENSING OPHTHALMIC DEVICE

(71) Applicant: Johnson & Johnson Vision Care, Inc., Jacksonville, FL (US)

(72) Inventors: Steven Hoggarth, Carey, NC (US); Randall B. Pugh, Jacksonville, FL (US); Adam Toner, Jacksonville, FL (US)

(21) Appl. No.: 15/458,286

(22) Filed: Mar. 14, 2017

(51) Int. Cl. G02C 11/00; G02C 7/04; G02C 7/08; A61F 9/007; A61B 5/01; A61B 5/00

(52) U.S. Cl. CPC G02C 11/10; G02C 7/049; G02C 7/083; A61F 9/007; A61B 5/4836; A61B 5/6803; A61B 5/7282; A61B 5/4306; A61B 5/01

(57) ABSTRACT

The present disclosure relates to sensor systems for electronic ophthalmic devices. In certain embodiments, the sensor systems may comprise a temperature sensor disposed adjacent an eye of a user, the temperature sensor configured to sense a temperature on or adjacent an eye of a wearer of the ophthalmic device, the temperature sensor further configured to provide an output indicative of the sensed temperature and a processor configured to receive the output and to determine a physiological characteristic of the user based at least on the output.

### BACKGROUND OF THE DISCLOSURE

### 1. Field of the Disclosure

The present disclosure relates to electronic ophthalmic devices, such as wearable lenses, including contact lenses, implantable lenses, including intraocular lenses (IOLs) and any other type of device comprising optical components, and more particularly, to sensors and associated hardware and software for sensing temperature at or near an eye of a user.

### 2. Discussion of the Related Art

Ophthalmic devices, such as contact lenses and intraocular lenses, currently are utilized to correct vision defects such as myopia (nearsightedness), hyperopia (farsightedness), presbyopia and astigmatism. However, properly designed lenses incorporating additional components may be utilized to enhance vision as well as to correct vision defects.

Conventional contact lenses are polymeric structures with specific shapes to correct various vision problems as briefly set forth above. However, enhanced functionality, beyond the correction of vision may be desirable. Accordingly, improvement of conventional ophthalmic devices is needed.

### SUMMARY OF THE DISCLOSURE

The present disclosure relates to powered or electronic ophthalmic devices that may comprise an electronic system. The electronic system includes one or more batteries or other power sources, power management circuitry, one or more sensors, clock generation circuitry, control algorithms, circuitry comprising a temperature sensor, and lens driver circuitry.

The present disclosure relates to electronic ophthalmic devices comprising one or more sensor systems described herein. In certain embodiments, an electronic ophthalmic device may comprise an ophthalmic lens having an optic zone and a peripheral zone. An ophthalmic device may comprise a variable optic element incorporated into the optic zone of the ophthalmic lens, the variable optic being configured to change the refractive power of the ophthalmic lens. An ophthalmic device may comprise an electronic component incorporated into the peripheral zone of the ophthalmic lens, the electronic component including the sensor system for detecting temperature on or adjacent the eye of a wearer.

The present disclosure relates to a sensing system comprising a temperature sensor disposed adjacent an eye of a user. The temperature sensor may be configured to sense a temperature on or adjacent an eye of a wearer of the ophthalmic device. The temperature sensor may be configured to provide an output indicative of the sensed temperature and a processor configured to receive the output and to determine a physiological characteristic of the user based at least on the output.

The present disclosure relates to an ophthalmic device comprising an ophthalmic lens having an optic zone and a peripheral zone and a sensor system disposed in the peripheral zone of the ophthalmic lens, the sensor system comprising a temperature sensor configured to sense a temperature on or adjacent an eye of a wearer of the ophthalmic device, the temperature sensor further configured to provide an output indicative of the sensed temperature.

The present disclosure relates to methods for determining a physiological characteristic of a user of an ophthalmic device. Methods may comprise receiving, via a temperature sensor disposed adjacent an eye of the user, a temperature signal indicative of a temperature on or adjacent the eye of the user. Methods may comprise determining, based at least on the temperature signal, a temperature signature indicative of the physiological characteristic of the user. Methods may further comprise implementing, via a controller, a predetermined function associated with the ophthalmic device.

### BRIEF DESCRIPTION OF THE DRAWINGS

The foregoing and other features and advantages of the disclosure will be apparent from the following, more particular description of preferred embodiments of the disclosure, as illustrated in the accompanying drawings.

FIG. 1 illustrates an exemplary ophthalmic device comprising a sensor system in accordance with some embodiments of the present disclosure.

FIG. 2 illustrates an exemplary ophthalmic device comprising a sensor system in accordance with some embodiments of the present disclosure.

FIG. 3 is a planar view of an ophthalmic device comprising electronic components, including a sensor system and a variable-optic element in accordance with the present disclosure.

FIG. 4 is a diagrammatic representation of an exemplary insert, including a sensor system, positioned in a powered or electronic ophthalmic device in accordance with some embodiments of the present disclosure.

FIG. 5A is a diagrammatic representation of an exemplary electronic system incorporated into a contact lens for detecting eyelid position in accordance with the present disclosure.

FIG. 5B is an enlarged view of the exemplary electronic system of FIG. 5A.

FIG. 6A is a diagrammatic representation of an exemplary sensor system incorporated into an ophthalmic device in accordance with the present disclosure.

FIG. 6B is an enlarged view of the exemplary sensor system of FIG. 6A.

### DETAILED DESCRIPTION

Ophthalmic devices may include wearable lenses (e.g., contact lenses), implantable lenses, including intraocular lenses (IOLs) and any other type of device comprising optical components. To achieve enhanced functionality, various circuits and components may be integrated into these ophthalmic devices. For example, control circuits, microprocessors, communication devices, power supplies, sensors, actuators, light-emitting diodes, and miniature antennas may be integrated into ophthalmic devices via custom-built optoelectronic components to not only correct vision, but to enhance vision as well as provide additional functionality as is explained herein. As an example, electronic and/or powered contact lenses may be designed to provide enhanced vision via zoom-in and zoom-out capabilities, or just simply modifying the refractive capabilities of the lenses. Electronic and/or powered contact lenses may be designed to enhance color and resolution, to display textural information, to translate speech into captions in real time, to offer visual cues from a navigation system, and to provide image processing and internet access. The lenses may be designed to allow the wearer to see in low light conditions. The properly designed electronics and/or arrangement of electronics on lenses may allow for projecting an image onto the retina, for example, without a variable focus optic lens, provide novelty image displays and even provide wakeup alerts. Alternately, or in addition to any of these functions or similar functions, the contact lenses may incorporate components for the noninvasive monitoring of the wearer's biomarkers and health indicators. For example, sensors built into the lenses may allow a diabetic patient to keep tabs on blood sugar levels by analyzing components of the tear film without the need for drawing blood. In addition, an appropriately configured lens may incorporate sensors for monitoring cholesterol, sodium, and potassium levels, as well as other biological markers. This coupled with a wireless data transmitter could allow a physician to have almost immediate access to a patient's blood chemistry without the need for the patient to waste time getting to a laboratory and having blood drawn. In addition, sensors built into the lenses may be utilized to detect light incident on the eye to compensate for ambient light conditions or for use in determining blink patterns.

The powered or electronic ophthalmic devices of the present disclosure may comprise the necessary elements to correct and/or enhance the vision of patients with one or more of the above described vision defects or otherwise perform a useful ophthalmic function. In addition, the electronic contact lens may be utilized simply to enhance normal vision or provide a wide variety of functionality as described above. The electronic contact lens may comprise a variable focus optic lens, an assembled front optic embedded into a contact lens or just simply embedding electronics without a lens for any suitable functionality. The electronic lens of the present disclosure may be incorporated into any number of contact lenses as described above. In addition, intraocular lenses may also incorporate the various components and functionality described herein. However, for ease of explanation, the disclosure will focus on an electronic contact lens to correct vision defects intended for single-use daily disposability.

The present disclosure may be employed in a powered ophthalmic lens or powered contact lens comprising an electronic system, which actuates a variable-focus optic or any other device or devices configured to (e.g., operable to) implement any number of numerous functions that may be performed. The electronic system includes one or more batteries or other power sources, power management circuitry, one or more sensors, clock generation circuitry, control algorithms and circuitry, and lens driver circuitry. The complexity of these components may vary depending on the required or desired functionality of the lens.

Control of an electronic or a powered ophthalmic lens may be accomplished through a manually operated external device that communicates with the lens, such as a hand-held remote unit. For example, a fob may wirelessly communicate with the powered lens based upon manual input from the wearer. Alternately, control of the powered ophthalmic lens may be accomplished via feedback or control signals directly from the wearer.

The eye comprises a number of liquid components, including the tear film. These liquids are excellent conductors of electrical signals as well as other signals, such as acoustic signals or sound waves. Accordingly, it should be understood that a temperature sensor in accordance with the present disclosure may provide feedback signals for controlling any number of functions that may be implemented by a powered or electronic ophthalmic lens.

A sensor, the components of which may be embedded in an ophthalmic device such as a powered contact lens, may detect characteristics (e.g., physiological characteristics) of a user. For example, a temperature sensor may be disposed adjacent an eye of a user and configured to (e.g., operable to) detect a temperature on or adjacent the eye. The temperature sensor may provide an output indicative of the detected temperature. The temperature sensor may be configured to (e.g., operable to) detect an absolute temperature and/or a relative temperature. As an example, the temperature sensor may be activated or initialized and may determine a base reference temperature at or during initialization. Subsequent temperature detection may be relative to the base reference temperature and may indicate a temperature change (delta) relative to the base reference temperature. The output of the temperature sensor may be transmitted to a processor/controller, which may be disposed adjacent the ophthalmic device or spaced therefrom. As such, the processor may determine a physiological characteristic of the user based at least on the output of the temperature sensor. The physiological characteristic may indicate fertility and/or a medical condition such as a disease. The processor/controller may be configured to (e.g., operable to) cause execution of a predetermined function such as release of a treatment adjacent the eye of the user.

Sensors may comprise a non-contact sensor, such as an antenna that is embedded into a contact lens or other ophthalmic device, but that does not directly touch the surface of an eye. Alternately, sensors may comprise a contact sensor, such as contact pads that directly touch the surface of an eye. It is important to note that any number of suitable devices and processes may be utilized for the detection of temperature, for example, thermocouples. As described herein, any type of sensor and/or sensing technology may be utilized.

In certain embodiments, ophthalmic devices may comprise an ophthalmic lens having an optic zone and a peripheral zone. Ophthalmic devices may comprise a variable optic element incorporated into the optic zone of the ophthalmic lens, the variable optic being configured to (e.g., operable to) change the refractive power of the wearable ophthalmic lens. Ophthalmic devices may comprise a sensor system disposed in the peripheral zone of the ophthalmic lens, the sensor system comprising a temperature sensor configured to (e.g., operable to) sense a temperature on or adjacent an eye of a wearer of the ophthalmic device, the temperature sensor further configured to (e.g., operable to) provide an output indicative of the sensed temperature.

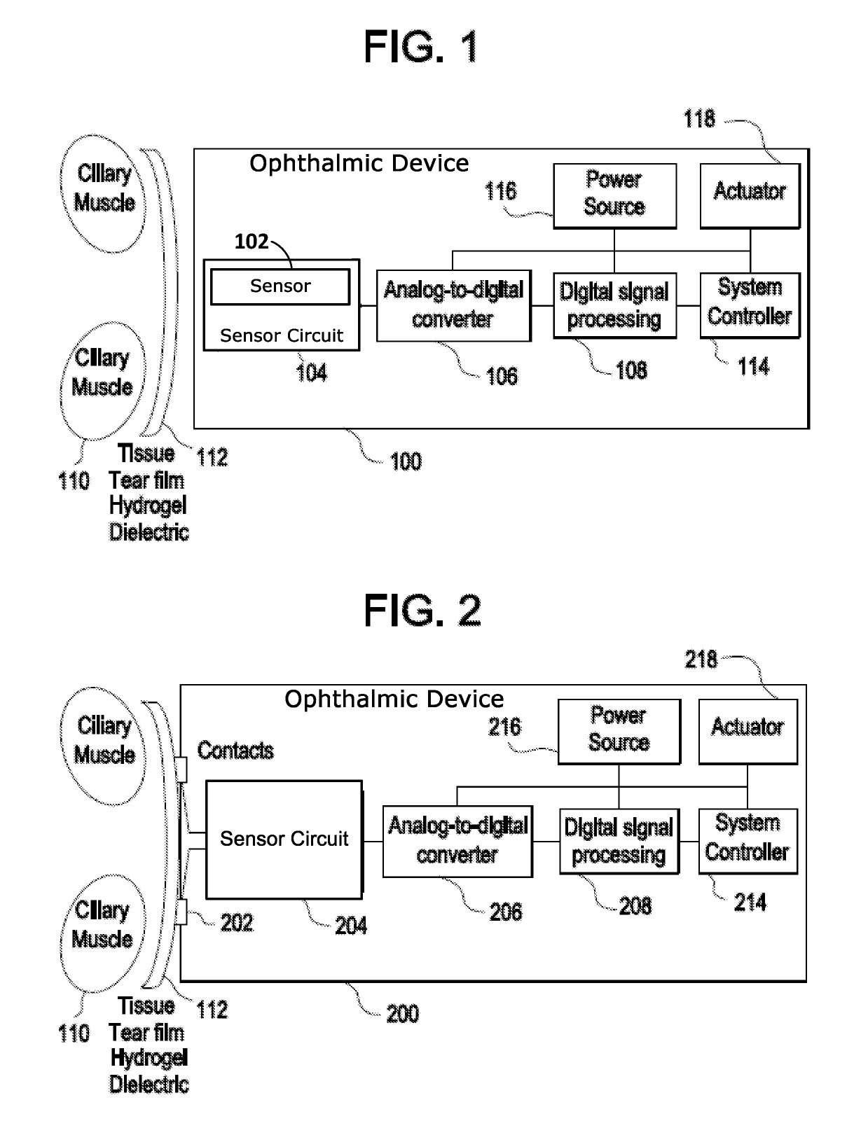

FIG. 1 illustrates, in block diagram form, an ophthalmic device 100 disposed on the front surface of the eye or cornea 112, in accordance with one exemplary embodiment of the present disclosure. Although the ophthalmic device 100 is shown and described as a being disposed on the front surface of the eye, it is understood that other configurations, such as those including intraocular lens configuration may be used. In this exemplary embodiment, the sensor system may comprise one or more of a sensor 102, a sensor circuit 104, an analog-to-digital converter 106, a digital signal processor 108, a power source 116, an actuator 118, and a system controller 114. As illustrated, the ciliary muscle 110 is located behind the front eye surface or cornea 112. Although not shown, it is understood that the eye comprises additional anatomical components including, but not limited to, iris, vitreous humor, retina, sclera, blood vessel, etc. As set forth above, the various fluids comprising the eye are good conductors of electrical and acoustical signals. Further, the thermal properties of the eye have been studied in the art, an example of which is illustrated in Table 1:

TABLE 1
Thermal properties of various parts of human eye*

Property

| | Thermal | Specific heat | |
|---|---|---|---|
| | conductivity | capacity | Density |
| Eye tissue | K(Wm⁻¹K⁻¹) | C (JKg⁻¹K⁻¹) | ρ(kgm⁻³) |
| Cornea | 0.580 | 4178 | 1050 |
| Aqueous humor | 0.578 | 3997 | 7 1050 |
| Lens | 0.400 | 3000 | 1000 |
| Iris | 1.680 | 3650 | 1100 |
| Vitreous humor | 0.594 | 3997 | 1000 |
| Retina | 0.565 | 3680 | 1000 |
| Sclera | 0.580 | 4178 | 1000 |
| Blood | 0.530 | 3600 | 1050 |

*Journal of Lasers in Medical Science (2013) Autumn; 4(4): 175-181, citing Narasimhan A, Jha K K. Bio-heat transfer simulation of square and circular array of retinal laser irradiation. Front Heat Mass Transfer. 2010; 53: 482-90, and Cvetkovic M, Poljak D, Pretta A. Thermal Modeling of the Human Eye Exposed to Laser Radiation. IEEE SoftCOM 2008. 16th Int. Con., September 2008.

The properties reported in Table 1 are shown as an example of heat transfer modelling in a human eye. However, the specific properties reported are not intended to be limiting to the scope of the devices, systems, and methods disclosed herein. Instead, such modelling illustrates that a temperature measurement on or adjacent the eye may be correlated to a temperature elsewhere in the body, such as a core body temperature. As such, temperature detected on and/or adjacent the eye may be indicative of a physiological characteristic of a user. Such characteristic may comprise a core body temperature or a change in core body temperature. Moreover, the detected temperature may be indicative of fertility and/or a medical condition such as a disease.

In this exemplary embodiment, the sensor 102 may be at least partially embedded into the ophthalmic device 100. The sensor 102 may be in thermal communication with the eye, for example, disposed to sense temperature change associated with heat translating through the eye. The sensor 102 may be or comprise one or more components configured to sense a temperature at or near the eye. The sensor 102 may be configured to generate an electrical signal indicative of the sensed temperature. As such, when thermal characteristics of the user change, the sensor 102 may sense absolute temperature, relative temperature, or temperature change due to such thermal characteristic and may generate the electrical signal indicative of such change or resultant characteristic. For example, there may be various signals detected by the sensor 102. As a further example, a set of temperature signatures may be determined (e.g., via experimentation) and may be stored for subsequent comparison. Periodic temperature samples may be detected over a time period in order to determine thermal noise such as ambient temperature noise and or natural variability in a particularly user's temperature.

As an example, a fertility signature may be determined based on a plurality of temperature measurements over a period of time. Over time, a woman's basal body temperature may fluctuate during a follicular phase of a menstrual cycle. During this time, a cover line temperature may be established as a base reference temperature. Such a time period may be predetermined for a particular user and may be adjusted. When the basal body temperature drops from the base reference temperature by a predetermined threshold amount (e.g., 0.2° C., 0.3° C., 0.4° C., etc.), the change in temperature may be indicative of ovulation. When the basal body temperature is elevated by a predetermined threshold amount (e.g., 0.2° C., 0.3° C., 0.4° C., etc.), the change may be indicative of the luteal phase. As such, similar eye temperature measurements may be sampled over a period of time and a fertility signature correlating to a basal body temperature may be developed. In this way, the fertility signature may be stored and referenced against subsequent temperature measurements to determine a state in a woman's menstrual cycle. As a further example, a fever signature or disease signature may be determined by sampling temperature over a period of time and comparing one or more changes in temperature to a predetermined temperature signature indicative of a physiological characteristic such as a medical condition.

In certain aspects, a plurality of ophthalmic devices (e.g., ophthalmic devices 100) may each comprise at least one temperature sensor such as sensor 102. A first ophthalmic device may be disposed adjacent an eye of a user. As such, temperature measurements detected by a first sensor associated with the first ophthalmic device may be stored for subsequent reference. Such storage may comprise transmitting sensor measurement information from the ophthalmic device to a storage spaced from the ophthalmic device. As an example, a transmitter may be configured to transmit the sensor measurement information via a radio signal, optical signal, or the like to a remote storage device. The first ophthalmic device may be removed from the eye (e.g., disposal contact lens). A second ophthalmic device may be disposed adjacent the eye of the user. As such, a second sensor associated with the second ophthalmic device may detect temperature measurements. The temperature measurements captured via the second sensor may be processed with the stored temperature measurements to determine temperature characteristics relating to the user across multiple lenses.

The sensor circuit 104 or sensor system may be configured to process signals received by the sensor 102. As an example, the sensor circuit 104 may be configured to amplify a signal to facilitate integration of small changes in signal level. As a further example, the sensor circuit 104 may be configured to amplify a signal to a useable level for the remainder of the system, such as giving a signal enough power to be acquired by various components of the sensor circuit 104 and/or the analog-to-digital converter 106. In addition to providing gain, the sensor circuit 104 may include other analog signal conditioning circuitry such as filtering and impedance matching circuitry appropriate to the sensor 102 and sensor circuit 104 output. The sensor circuit 104 may comprise any suitable device for amplifying and conditioning the signal output by the sensor 102. For example, the sensor circuit 104 may simply comprise a single operational amplifier or a more complicated circuit comprising one or more operational amplifiers.

As set forth above, the sensor 102 and the sensor circuit 104 are configured to capture and isolate the signals indicative of eye temperature from the noise and other signals (e.g., ambient temperature shifts) affecting the eye, and convert it to a signal usable ultimately by the system controller 114. The system controller 114 is preferably preprogrammed to recognize the various temperature signatures under various conditions and provide an appropriate output signal to the actuator 118.

In this exemplary embodiment, the analog-to-digital converter 106 may be used to convert an analog signal output from the amplifier into a digital signal for processing. For example, the analog-to-digital converter 106 may convert an analog signal output from the sensor circuit 104 into a digital signal that may be useable by subsequent or downstream circuits, such as a digital signal processing system 108 or microprocessor. A digital signal processing system or digital signal processor 108 may be utilized for digital signal processing, including one or more of filtering, processing, detecting, and otherwise manipulating/processing sampled data to discern eye temperature from noise and interference. The digital signal processor 108 may be preprogrammed with the temperature signatures described herein. The digital signal processor 108 may be implemented utilizing analog circuitry, digital circuitry, software and/or preferably a combination thereof.

A power source 116 supplies power for numerous components comprising the non-contact sensor system. The power may be supplied from a battery, energy harvester, or other suitable means as is known to one of ordinary skill in the art. Essentially, any type of power source may be utilized to provide reliable power for all other components of the system. A certain temperature or temperature signature, processed from analog to digital, may enable activation of the system controller 114. Furthermore, the system controller 114 may control other aspects of a powered contact lens depending on input from the digital signal processor 108, for example, changing the focus or refractive power of an electronically controlled lens through an actuator 118, or causing release of a treatment.

FIG. 2 illustrates an ophthalmic device 200, comprising a sensor system, shown on the front surface of the eye or cornea 112 in accordance with another exemplary embodiment of the present disclosure. In this exemplary embodiment, a sensor system may comprise a contact or multiple contacts 202, a sensor circuit 204, an analog-to-digital converter 206, a digital signal processor 208, a power source 216, an actuator 218, and a system controller 214. The ciliary muscle 110 is located behind the front eye surface or cornea 112. The ophthalmic device 200 is placed onto the front surface of the eye 112, such that the electronic circuitry of the sensor may be utilized to implement the neuromuscular sensing of the present disclosure. The components of this exemplary system are similar to and perform the same functions as those illustrated in FIG. 1, with the exception of contacts 202 and the sensor circuit 204. In other words, since direct contacts 202 are utilized, there is no need for an antenna or an amplifier to amplify and condition the signal received by the antenna.

In the illustrated exemplary embodiment, the contacts 202 may provide for a direct electrical connection to the tear film and the eye surface. For example, the contacts 202 may be implemented as metal contacts that are exposed on the back curve of the ophthalmic device 200 and be made of biocompatible thermally conductive materials. Furthermore, the contact lens polymer may be molded around the contacts 202, which may aid in comfort on the eye and provide improved conductivity through the ophthalmic device 200. Additionally, the contacts 202 may provide for a low resistance connection between the eye's surface 112 and the electronic circuitry within the ophthalmic device 200. Four-terminal sensing, also known as Kelvin sensing, may be utilized to mitigate contact resistance effects on the eye. The sensor circuit 204 may emit a signal with several constituent frequencies or a frequency sweep, while measuring the voltage/current across the contacts 202.

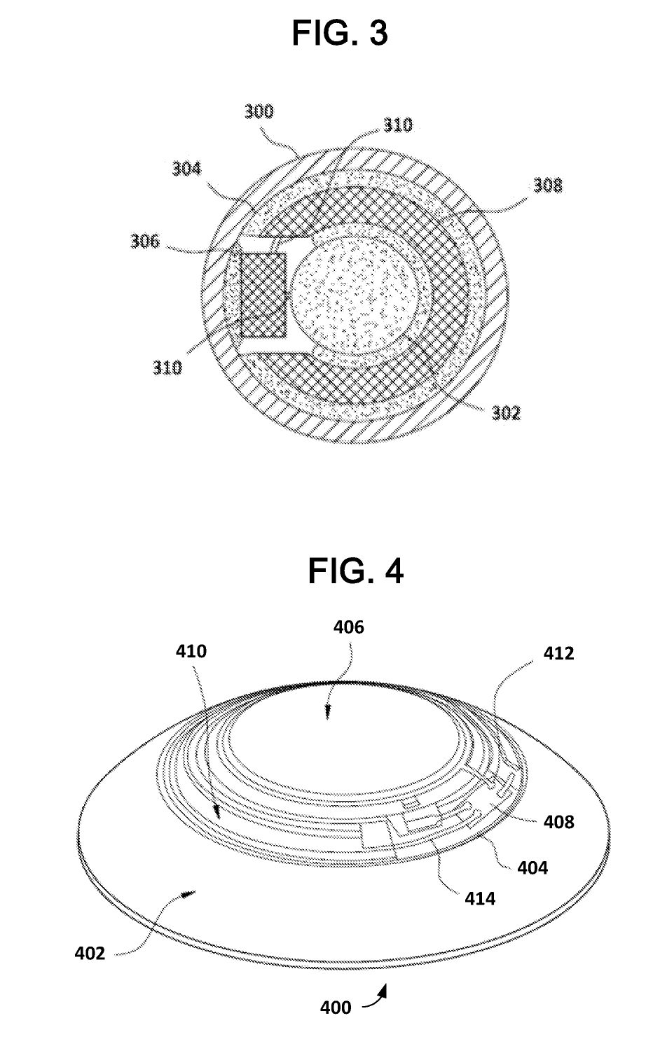

Referring now to FIG. 3, there is illustrated, in planar view, a wearable electronic ophthalmic device comprising a sensor in accordance with the present disclosure. The ophthalmic device 300 comprises an optic zone 302 and a peripheral zone 304. The optic zone 302 may function to provide one or more of vision correction, vision enhancement, other vision-related functionality, mechanical support, or even a void to permit clear vision. In accordance with the present disclosure, the optic zone 302 may comprise a variable optic element configured to provide enhanced vision at near and distant ranges. The variable-optic element may comprise any suitable device for changing the focal length of the lens or the refractive power of the lens. For example, the variable optic element may be as simple as a piece of optical grade plastic incorporated into the lens with the ability to have its spherical curvature changed. The peripheral zone 304 comprises one or more of electrical circuits 306, a power source 308, electrical interconnects 310, mechanical support, as well as other functional elements.

The electrical circuits 306 may comprise one or more integrated circuit die, printed electronic circuits, electrical interconnects, and/or any other suitable devices, including the sensing circuitry described herein. The power source 308 may comprise one or more of battery, energy harvesting, and or any other suitable energy storage or generation devices. It is readily apparent to the skilled artisan that FIG. 3 only represents one exemplary embodiment of an electronic ophthalmic lens and other geometrical arrangements beyond those illustrated may be utilized to optimize area, volume, functionality, runtime, shelf life as well as other design parameters. It is important to note that with any type of variable optic, the fail-safe is distance vision. For example, if power were to be lost or if the electronics fail, the wearer is left with an optic that allows for distance vision. In certain aspects, the temperature measurements determined using the sensing circuitry (e.g., sensors) associated with the electrical circuits 306 may be used to cause a reconfiguration of the variable optic element. As an example, certain temperature measurements or temperature changes may cause a change in focal length of the lens or a change in refractive power.

FIG. 4 is a diagrammatic representation of an exemplary electronic insert, including a sensor system, positioned in a powered or electronic ophthalmic device in accordance with the present disclosure. As shown, a contact lens 400 comprises a soft plastic portion 402 which comprises an electronic insert 404. This insert 404 includes a lens 406 which is activated by the electronics, for example, focusing near or far depending on activation. Integrated circuit 408 mounts onto the insert 404 and connects to batteries 410, lens 406, and other components as necessary for the system. The integrated circuit 408 includes a sensor 412 and associated signal path circuits. The sensor 412 may comprise any sensor configuration such as those described herein. The sensor 412 may also be implemented as a separate device mounted on the insert 404 and connected with wiring traces 414.

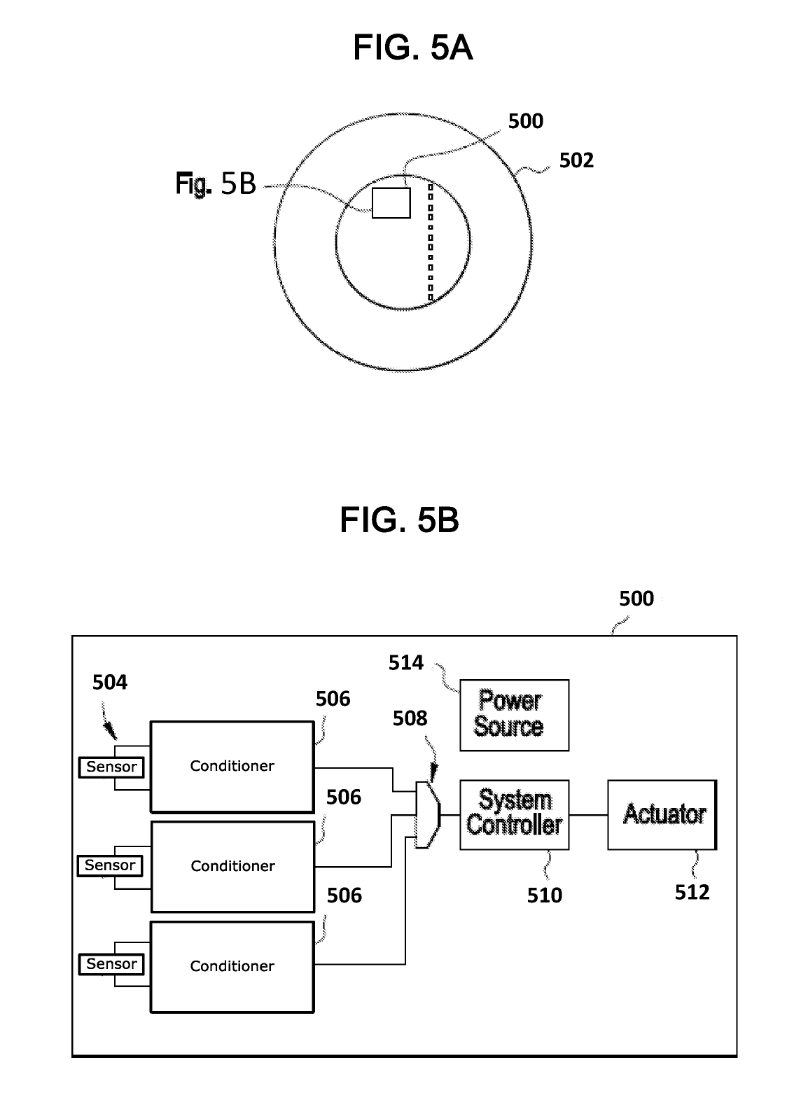

FIGS. 5A and 5B illustrate an alternate exemplary detection system 500 incorporated into an ophthalmic device 502 such as a contact lens. FIG. 5A shows the system 500 on the device 502 and FIG. 5B shows an exemplary schematic view of the system 500. The system 500 may be a blink or eyelid position detection system that comprises multiple sensors to determine the position of the eyelids. These sensors may comprise outward facing light detectors. In this exemplary embodiment, temperature sensors 504 may be used to sense a temperature at and/or adjacent an eye of the user of the ophthalmic device 502.

As an illustrate example, the temperature sensors 504 and/or the temperature sensors described herein relating to various aspects may be or comprise a sensor having the following configurations illustrated in Table 2:

TABLE 2

| Parameter | Example Performance Target | |
|---|---|---|
| Accuracy | .1-.5° C. or .5° F. | |
| Speed | 10 | μs |
| Temperature Range | 75-105° | F. |
| Operating Voltage | 1.0-1.5 | V |

| | |
|---|---|
| Power | 20 μW Active |

It is understood that the configurations illustrated in Table 2 are examples only and are not limiting. As a further example, the sensors 504 may be configured to sense a temperature invariant voltage and a voltage that is configured to respond contrary to absolute temperature. A difference between the two voltages may represent a bandgap reference, which may be amplified and digitized as a output of the sensors 504.

Sensor conditioners 506 create an output signal indicative of a measurement of one or more sensors 504 in communication with a respective one or more of the sensor conditioners 506. For example, the sensor conditioners may amplify and or filter a signal received from a respective sensor 504. The output of the sensor conditioners 506 may be combined with a multiplexer 508 to reduce downstream circuitry.

In certain embodiments, downstream circuitry may include a system controller 510, which may comprise an analog-to-digital converter (ADC) that may be used to convert a continuous, analog signal into a sampled, digital signal appropriate for further signal processing. For example, the ADC may convert an analog signal into a digital signal that may be useable by subsequent or downstream circuits, such as a digital signal processing system or microprocessor, which may be part of the system controller 510 circuit. A digital signal processing system or digital signal processor may be utilized for digital signal processing, including one or more of filtering, processing, detecting, and otherwise manipulating/processing sampled data. The digital signal processor may be preprogrammed with various displacement signatures. As an example, a data store of temperature measurements or signatures may be mapped to particular user conditions having particular physiological characteristics. As such, when temperature measurements matching or near a particular signature are detected, the associated physiological characteristic or user condition may be extrapolated. The digital signal processor also comprises associated memory. The digital signal processor may be implemented utilizing analog circuitry, digital circuitry, software, and/or preferably a combination thereof.

The system controller 510 receives inputs from the sensor conditioner 506 via a multiplexor 508, for example, by activating each sensor 504 in order and recording the values. It may then compare measured values to pre-programmed patterns and historical samples to determine a temperature patterns, characteristics and signatures. It may then activate a function in an actuator 512, for example, causing a treatment to be released into the eye. The sensors 504, and/or the whole electronic system, may be encapsulated and insulated from the saline contact lens environment. Various configurations of the sensors 504 may facilitate optimal sensing conditions as the ophthalmic device 502 shifts or rotates.

A power source 514 supplies power for numerous components comprising the lid position sensor system 500. The power source 514 may also be utilized to supply power to other devices on the contact lens. The power may be supplied from a battery, energy harvester, or other suitable means as is known to one of ordinary skill in the art. Essentially, any type of power source 514 may be utilized to provide reliable power for all other components of the system. A temperature sensor array pattern, processed from analog to digital, may enable activation of the system controller 510 or a portion of the system controller 510. Furthermore, the system controller 510 may control other aspects of a powered contact lens depending on input from the multiplexor 508, for example, changing the focus or refractive power of an electronically controlled lens through the actuator 512.

In one exemplary embodiment, the electronics and electronic interconnections are made in the peripheral zone of a contact lens rather than in the optic zone. In accordance with an alternate exemplary embodiment, it is important to note that the positioning of the electronics need not be limited to the peripheral zone of the contact lens. All of the electronic components described herein may be fabricated utilizing thin film technology and/or transparent materials. If these technologies are utilized, the electronic components may be placed in any suitable location as long as they are compatible with the optics. The activities of the digital signal processing block and system controller (system controller 510 in FIG. 5B) depend on the available sensor inputs, the environment, and user reactions. The inputs, reactions, and decision thresholds may be determined from one or more of ophthalmic research, preprogramming, training, and adaptive/learning algorithms. For example, the general thermal modelling of a human eye may be documented in literature, applicable to a broad population of users, and pre-programmed into system controller. However, an individual's deviations from the general expected response may be recorded in a training session or part of an adaptive/learning algorithm which continues to refine the response in operation of the electronic ophthalmic device. In one exemplary embodiment, the user may train the device by activating a handheld fob, which communicates with the device, when the user desires near focus. A learning algorithm in the device may then reference sensor inputs in memory before and after the fob signal to refine internal decision algorithms. This training period could last for one day, after which the device would operate autonomously with only sensor inputs and not require the fob.

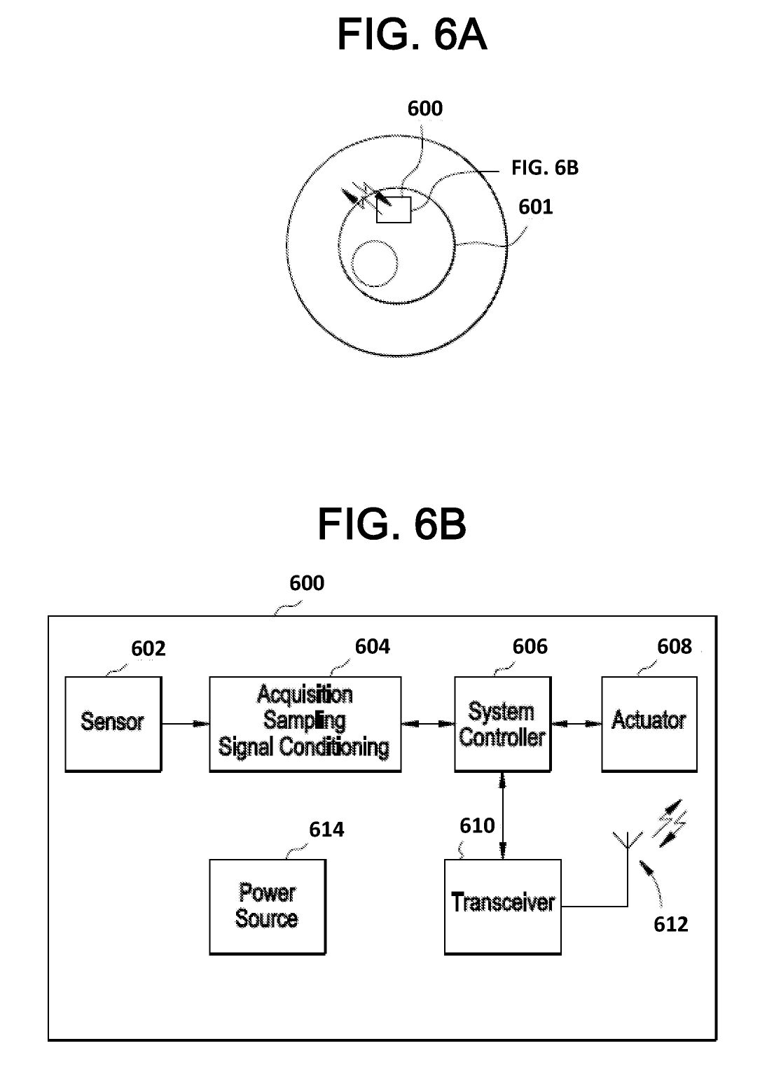

FIGS. 6A and 6B are diagrammatic representations of an exemplary pupil position and convergence detection system 600 for control of one or more aspects of a powered ophthalmic lens. Sensor 602 detects the movement and/or position of the pupil or, more generally, the eye. The sensor 602 may be implemented as a multi-axis accelerometer on a contact lens 601. Such sensors 602 may be used in conjunction with the temperature sensors described herein. With the contact lens 601 being affixed to the eye and generally moving with the eye, an accelerometer on the contact lens 601 may track eye movement. The sensor 602 may also be implemented as a rear-facing camera or sensor which detects changes in images, patterns, or contrast to track eye movement. Alternately, the sensor 602 may comprise neuromuscular sensors to detect nerve and/or muscle activity which moves the eye in the socket. There are six muscles attached to each eye globe which provide each eye with a full range of movement and each muscle has its own unique action or actions. These six muscles are innervated by one of the three cranial nerves. It is important to note that any suitable device may be utilized as the sensor 602, and more than a single sensor 602 may be utilized. The output of the sensor 602 is acquired, sampled, and conditioned by signal processor 604. The signal processor 604 may include any number of devices including an amplifier, a transimpedance amplifier, an analog-to-digital converter, a filter, a digital signal processor, and related circuitry to receive data from the sensor 602 and generate output in a suitable format for the remainder of the components of the system 600. The signal processor 604 may be implemented utilizing analog circuitry, digital circuitry, software, and/or preferably a combination thereof. It should be appreciated that the signal processor 604 is co-designed with the sensor 602 utilizing methods that are known in the relevant art, for example, circuitry for acquisition and conditioning of an accelerometer are different than the circuitry for a muscle activity sensor or optical pupil tracker. The output of the signal processor 604 is preferentially a sampled digital stream and may include absolute or relative position, movement, detected gaze in agreement with convergence, or other data. System controller 606 receives input from the signal processor 604 and uses this information, in conjunction with other inputs, to control the electronic contact lens 601. For example, the system controller 606 may output a signal to an actuator 608 that controls a variable power optic in the contact lens 601. If, for example, the contact lens 601 is currently in a far focus state and the sensor 602 detects convergence, the system controller 606 may command the actuator 608 to change to a near focus state. System controller 606 may both trigger the activity of sensor 602 and the signal processor 604 while receiving output from them. A transceiver 610 receives and/or transmits communication through antenna 612. This communication may come from an adjacent contact lens, spectacle lenses, or other devices. The transceiver 610 may be configured for two-way communication with the system controller 606. Transceiver 610 may contain filtering, amplification, detection, and processing circuitry as is common in transceivers. The specific details of the transceiver 610 are tailored for an electronic or powered contact lens, for example the communication may be at the appropriate frequency, amplitude, and format for reliable communication between eyes, low power consumption, and to meet regulatory requirements.

Transceiver 610 and antenna 612 may work in the radio frequency (RF) bands, for example 2.4 GHz, or may use light for communication. However, other mechanisms of transmission such as optical communication may be used. Information received from transceiver 610 is input to the system controller 606, for example, information from an adjacent lens which indicates temperature measurements, convergence, or divergence. System controller 606 uses input data from the signal processor 604 and/or transceiver 610 to decide if a change in system state is required. The system controller 606 may also transmit data to the transceiver 610, which then transmits data over the communication link, for example via antenna 612. Although an antenna 612 is referenced, other communication mechanisms may be used such as an optical output (e.g., light source). The system controller 606 may be implemented as a state machine, on a field-programmable gate array, in a microcontroller, or in any other suitable device. Power for the system 600 and components described herein is supplied by a power source 614, which may include a battery, energy harvester, or similar device as is known to one of ordinary skill in the art. The power source 614 may also be utilized to supply power to other devices on the contact lens 601. The exemplary pupil position and convergence detection system 600 of the present disclosure is incorporated and/or otherwise encapsulated and insulated from the saline contact lens 601 environment.

In one exemplary embodiment, the electronics and electronic interconnections are made in the peripheral zone of a contact lens rather than in the optic zone. In accordance with an alternate exemplary embodiment, it is important to note that the positioning of the electronics need not be limited to the peripheral zone of the contact lens. All of the electronic components described herein may be fabricated utilizing thin film technology and/or transparent materials. If these technologies are utilized, the electronic components may be placed in any suitable location as long as they are compatible with the optics.

The activities of the acquisition sampling signal processing block and system controller (604 and 606 in FIG. 6B, respectively) depend on the available sensor inputs, the environment, and user reactions. The inputs, reactions, and decision thresholds may be determined from one or more of ophthalmic research, preprogramming, training, and adaptive/learning algorithms. For example, the general characteristics of eye movement may be well-documented in literature, applicable to a broad population of users, and pre-programmed into system controller. However, an individual's deviations from the general expected response may be recorded in a training session or part of an adaptive/learning algorithm which continues to refine the response in operation of the electronic ophthalmic device. In one exemplary embodiment, the user may train the device by activating a handheld fob, which communicates with the device, when the user desires near focus. A learning algorithm in the device may then reference sensor inputs in memory before and after the fob signal to refine internal decision algorithms. This training period could last for one day, after which the device would operate autonomously with only sensor inputs and not require the fob. An intraocular lens or IOL is a lens that is implanted in the eye and replaces the crystalline lens. It may be utilized for individuals with cataracts or simply to treat various refractive errors. An IOL typically comprises a small plastic lens with plastic side struts called haptics to hold the lens in position within the capsular bag in the eye. Any of the electronics and/or components described herein may be incorporated into IOLs in a manner similar to that of contact lenses.

Although shown and described in what is believed to be the most practical and preferred embodiments, it is apparent that departures from specific designs and methods described and shown will suggest themselves to those skilled in the art and may be used without departing from the spirit and scope of the disclosure. The present disclosure is not restricted to the particular constructions described and illustrated, but should be constructed to cohere with all modifications that may fall within the scope of the appended claims.

What is claimed is:

1. An ophthalmic device comprising:
an ophthalmic lens having an optic zone and a peripheral zone; and
a sensor system disposed in the peripheral zone of the ophthalmic lens, the sensor system comprising a temperature sensor configured to sense a temperature on or adjacent an eye of a wearer of the ophthalmic device, the temperature sensor further configured to provide an output indicative of the sensed temperature.

2. The ophthalmic device according to claim 1, wherein the ophthalmic lens comprises a contact lens.

3. The ophthalmic device according to claim 2, wherein the contact lens comprises a soft or hybrid contact lens.

4. The ophthalmic device according to claim 1, wherein the temperature sensor comprises one or more contacts configured to make direct contact with tear film of the eye.

5. The ophthalmic device according to claim 1, wherein the temperature sensor is configured to determine a reference temperature and a temperate change relative to the reference temperature.

6. The ophthalmic device according to claim 5, where the reference temperature is determined within an initialization time period associated with activation of the sensor system.

7. The ophthalmic device according to claim 1, wherein the temperature sensor is configured to determine an absolute temperature.

8. The ophthalmic device according to claim 1, further comprising a variable optic element incorporated into the optic zone of the ophthalmic lens, the variable optic element being configured to change the refractive power of the wearable ophthalmic lens.

9. The ophthalmic device according to claim 1, wherein the sensor system comprises a processor configured to receive the output and to determine a physiological characteristic of the user based at least on the output.

10. The ophthalmic device according to claim 9, wherein the physiological characteristic comprises an indication of fertility.

11. The ophthalmic device according to claim 9, wherein the physiological characteristic comprises an indication of a medical condition.

12. The ophthalmic device according to claim 11, wherein the medical condition comprises an indication of disease.

13. A sensor system for an ophthalmic device, the sensor system comprising:
a temperature sensor disposed adjacent an eye of a user, the temperature sensor configured to sense a temperature on or adjacent an eye of a wearer of the ophthalmic device, the temperature sensor further configured to provide an output indicative of the sensed temperature; and
a processor configured to receive the output and to determine a physiological characteristic of the user based at least on the output.

14. The sensor system according to claim 13, wherein the temperature sensor comprises one or more contacts configured to make direct contact with tear film of the eye.

15. The sensor system according to claim 13, wherein the temperature sensor is configured to determine a reference temperature and a temperate change relative to the reference temperature.

16. The sensor system according to claim 15, where the reference temperature is determined within an initialization time period associated with activation of the sensor system.

17. The sensor system according to claim 13, wherein the temperature sensor is configured to determine an absolute temperature.

18. The sensor system according to claim 13, further comprising a power source in electrical communication with one or more of the temperature sensor and the processor.

19. The sensor system according to claim 13, wherein the power source comprises a battery.

20. The sensor system according to claim 13, wherein the physiological characteristic comprises an indication of fertility.

21. The sensor system according to claim 13, wherein the physiological characteristic comprises an indication of a medical condition.

22. The sensor system according to claim 21, wherein the medical condition comprises an indication of disease.

23. A method for determining a physiological characteristic of a user of an ophthalmic device, the method comprising:
receiving, via a temperature sensor disposed adjacent an eye of the user, a temperature signal indicative of a temperature on or adjacent the eye of the user; and
determining, based at least on the temperature signal, a temperature signature indicative of the physiological characteristic of the user.

24. The method of claim 23, further comprising implementing, via a controller, a predetermined function associated with the ophthalmic device.

25. The method of claim 24, wherein the controller is disposed adjacent the eye of the user.

26. The method of claim 24, wherein the predetermined function comprises causing a treatment to be released on or adjacent the eye of the user.
